(19) United States
(12) Patent Application Publication
(10) Pub. No.: US 2018/0001225 A1
(43) Pub. Date: Jan. 4, 2018

(54) PLANT AND PROCESS FOR PRODUCING PURIFIED METHANOL

(71) Applicant: L'Air Liquide, Societe Anonyme pour I'Etude et I'Exploitation des Procedes Georges Claude, Paris (FR)

(72) Inventors: Timm SCHUHMANN, Offenbach (DE); Veronika GRONEMANN, Karben (DE)

(73) Assignee: L'Air Liquide, Societe Anonyme pour I'Etude et I'Exploitation des Procedes Georges Claude, Paris (FR)

(21) Appl. No.: 15/547,638

(22) PCT Filed: Feb. 17, 2016
(86) PCT No.: PCT/EP2016/025016

§ 371 Date: Jul. 31, 2017

(30) Foreign Application Priority Data
Feb. 24, 2015 (DE) 10 2015 102 627.9

(51) Int. Cl. B01D 3/14; C07C 29/80; B01D 3/42

(52) U.S. Cl. CPC B01D 3/148; B01D 3/4205; C07C 29/80

(57) ABSTRACT

The present invention relates to a plant for removing C₆-C₁₁ hydrocarbons from methanol, comprising at least one reactor for the conversion of carbon monoxide and hydrogen to a crude methanol containing hydrocarbons, a distillation column with a head and a sump for the purification of the methanol, and at least one conduit for guiding the crude methanol from the at least one reactor into the distillation column. At its head, the distillation column includes a feed conduit for feeding in water.

This invention relates to a plant for removing C₆-C₁₁ hydrocarbons from methanol, comprising at least one reactor for the conversion of carbon monoxide and hydrogen to a crude methanol containing hydrocarbons, a distillation column with a head and a sump for the purification of the methanol, and at least one conduit for guiding the crude methanol from the reactor into the distillation column. Equally, the invention also comprises a process for removing C₆-C₁₁ hydrocarbons from methanol.

Methanol (MeOH) is an organic chemical compound with the empirical formula CH₄O and is the simplest representative from the substance group of alcohols. Under normal conditions, methanol is a clear, colorless, flammable and readily volatile liquid with alcoholic odor. It mixes with many organic solvents and in any ratio with water.

With 45 million tons in annual production (status 2008), methanol is one of the organic chemicals most frequently produced. In the chemical industry it is above all used as starting substance for the production of formaldehyde, formic acid and acetic acid, to an increasing extent also as starting product for the production of olefins. The technical production of methanol chiefly is effected catalytically from carbon monoxide (CO) and hydrogen (H₂).

Such process for the production of methanol is known for example from EP 0 790 226 B1. The methanol is produced in a cyclic process in which a mixture of fresh and partly reacted synthesis gas first is supplied to a water-cooled reactor and then to a gas-cooled reactor, in each of which the synthesis gas is converted to methanol on a copper catalyst. The methanol produced in the process is separated from the synthesis gas to be recirculated, which then is countercurrently passed through the gas-cooled reactor as coolant and preheated to a temperature of 220 to 280° C., before it is introduced into the first synthesis reactor.

After its production, the crude methanol obtained must be purified, as it contains impurities due to insufficient purity of the educts and due to undesired side reactions.

GB 1,106,598 describes a treatment of crude methanol for removing iron carbonyl, in which the methanol to be purified is treated with oxygen or hydrogen peroxide in the presence of a substance of large surface area.

KR 10 2003 00 85 834 discloses a process for the purification of methanol, in which chlorine and styrene are removed by absorption on activated carbon.

JP 912 45 523 relates to the removal of zinc from methanol, which is effected by filtration and an ion exchanger.

US 2011/0306807 shows how methanol is obtained from a digester gas and purified. For this purpose, impurities first are condensed out, before the fraction containing methanol is distilled by addition of acid. By further distillation steps, the purity of the methanol then is increased further.

From WO 2010/091492 A1 a process for the purification of methanol is known, in which methanol is obtained as by-product of a sulfate efflux and subsequently purified. The purification is effected by distillation, wherein sulfuric acid is added to the distillation column to achieve a better separation result.

U.S. Pat. No. 3,434,937 describes a process for the purification of methanol with a focus on the separation of light alcohols, in particular ethanol, with three columns, wherein the methanol in the first column is withdrawn from the sump and in the second and third columns over head.

From U.S. Pat. No. 5,863,391 a process for removing acetaldehyde from methanol is known. For this purpose, a highly polar extracting agent such as glycerol or water is charged into an extractive column and withdrawn together with methanol, during which acetaldehyde can be separated reliably.

Such purification also is disclosed in GB 660,773. In this document, methanol is distilled by addition of water, in order to separate acetaldehyde and further alcohols.

All these processes, however, do not focus on the impurities in the form of longer-chain hydrocarbon compounds, in particular those with 6 to 11 C-atoms. These hydrocarbon fractions above all occur when iron impurities are contained in the synthesis gas and the educts thus undergo a Fischer-Tropsch synthesis as side reaction during the methanol synthesis. At very high hydrocarbon contents in the methanol, the impurity in the methanol becomes optically visible due to a turbidity even when it is diluted. At a dilution of 1:3 (crude methanol:water), the turbidity limit for a carbon fraction with ten C-atoms lies between 200 and 400 ppm.

It therefore is the object of the present invention to provide a process with which hydrocarbon fractions, in particular in the range of a chain length of six to eleven hydrocarbon atoms, can reliably be removed from the methanol, as above all in the case of a synthesis gas with low CO₂ content and hence little water in the crude methanol problems can occur with the separation of the hydrocarbons. Preferably, a purity required for the Grade AA specification should be achieved. According to US Spec O-M-232L for Grade AA methanol, the turbidity test must be passed with a 1:3 dilution with water.

This object is solved with a plant according to claim 1. Such plant for removing C₆-C₁₁ hydrocarbons from methanol comprises at least one reactor for the conversion of carbon monoxide and hydrogen to a crude methanol containing hydrocarbons. Connected to the reactor directly or indirectly, such plant furthermore contains a distillation column with a head and a sump for the purification of the methanol, wherein at least one conduit guides the crude methanol from the reactor into the distillation column. It is a feature essential for the invention that at its head the distillation column includes a feed conduit for feeding in water. It was found that by introducing water at the head of the column, the separation efficiency of the column with regard to contained hydrocarbons with six to eleven carbon atoms is improved distinctly.

In the sense of the invention, head of the column is understood to be that region which is arranged above the distillation region, which preferably is equipped with packings and/or trays. The sump in the sense of the invention is that region which is arranged below the distillation region of the column.

Preferably, such plant has a so-called flash evaporator or separator in the at least one conduit for guiding crude methanol from the reactor into the distillation column. By a simple phase separation, the separation of gases from the crude methanol thus can be effected.

What is furthermore preferred is an embodiment in which at least one scrubber is provided downstream of a flash evaporator, wherein this downstream arrangement is effected such that the scrubber is located in the gas discharge conduit of the flash evaporator. The gases separated by the flash evaporator thus can be purified.

In a particularly preferred embodiment of the invention the conduit for feeding water into the head of the distillation column is designed such that it guides the washing water from the at least one scrubber into the head of the distillation column. This has the advantage that no additional stream of water must be incorporated into the process, but an internal process stream can be employed. On the one hand, this simplifies the construction of the plant, as only one stream of water must be conducted. On the other hand, this also simplifies the purification of the stream of washing water, as a separate purification of the stream of washing water and the stream of water originating from the distillation column is omitted and only one single water treatment is necessary.

Continuing the invention, the distillation column is designed such that the purified methanol accumulates in the sump from which it is discharged. Such a distribution of the separation tasks has the advantage that all further fractions, in particular the hydrocarbons with six to eleven carbon atoms are withdrawn over head.

Furthermore, a preferred aspect of the invention provides that at its head the distillation column includes a reflux device with a heater and a condenser and the feed conduit for the water opens into the reflux device. This has the advantage that already by providing the heater, the water is brought to the temperature existing in the distillation column and there are no undesired breakthroughs of methanol, because at the introduction point of the water the temperature locally would decrease strongly and hence the separation efficiency also would be reduced distinctly.

In one aspect of the invention, the distillation column is a packed column. This is a usual form of column with which a uniform separation can be produced over the entire column.

In another embodiment of the invention, the column is a distillation column which includes sieve trays and/or bubble trays. By various trays, the separation tasks within the column thus can locally be solved by variation of the trays.

The invention furthermore also comprises a process for removing C₆-C₁₁ hydrocarbons from methanol with the features of claim 8. Such process comprises a conversion of carbon monoxide and hydrogen to a crude methanol containing hydrocarbons. Subsequently, this crude methanol is purified in a distillation column with a head and a sump. Water is added at the head of the column. By adding the water it is surprisingly found that the separation efficiency within the distillation column is improved distinctly with regard to contained hydrocarbons, in particular with a chain length of six to eleven carbon atoms.

What is preferred is an addition of 1 to 15 vol-%, particularly preferably 2 to 10 vol-% of water based on the total amount of the volume flow supplied via inflow and addition of water, as here a particularly good separation efficiency was found. Preferably, in one of 20 time units, preferably in one of 20 minutes during which the column was operated without interruption, the top product is recirculated into the column as reflux and otherwise discharged.

The separation efficiency is improved particularly when the hydrocarbons contained in the crude methanol include six to eleven carbon atoms in an amount of at least 50 vol-%, preferably at least 80 vol-%, based on the total volume flow of the hydrocarbons contained in the crude methanol.

Furthermore, the process according to the invention can be carried out particularly well in particular for crude methanol with a composition of between 90 and 97 vol-% of methanol, between 3 and 10 vol-% of water, up to 0.5 wt-% of hydrocarbons with a content of up to 15 wt-% of C₉₊ fraction and 0.3 wt-% of further impurities.

In a preferred aspect of the invention the distillation column includes a reflux, in order to achieve a clean separation of top and bottom product.

In a particularly preferred aspect, the reflux ratio lies between 1 and 5, as here a particularly good separation efficiency is achieved.

According to the invention, the distillation column is operated at a temperature between 75 and 100° C., preferably between 85 and 90° C. and/or at an absolute pressure of 1 to 3 bar. Methanol thereby can reliably be separated from hydrocarbons contained therein.

In a preferred embodiment of the invention, the flux withdrawn over head corresponds to 0.3 to 0.7 vol-% of the inflow of the column. By adjusting this ratio, the separation efficiency likewise can be optimized.

By locally increasing the water content in the head of the column, a local temperature increase is obtained there, as the quantity of the boiling components now is changed in relation to the total volume. This local temperature increase effects a shift in the distribution of the hydrocarbon fractions to be separated into the gas fraction of the column, whereby the separation efficiency is improved in an inventive way.

Further developments, advantages and possible applications of the invention can also be taken from the following description of the drawing and the exemplary embodiments. All features described and/or illustrated form the subject-matter of the invention per se or in any combination, independent of their inclusion in the claims or their back-references.

In the drawing:

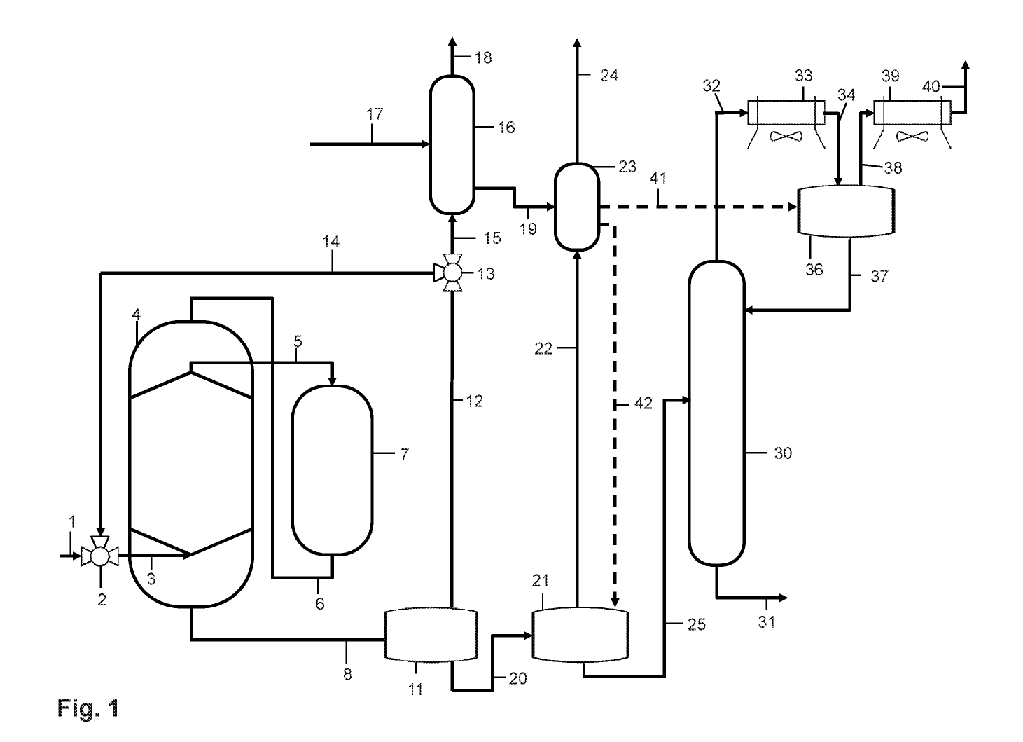

FIG. 1 shows a plant according to the invention for removing C₆-C₁₁ hydrocarbons from methanol with a column designed according to the invention.

FIG. 1 shows the flow diagram of a plant or a process for removing C₆-C₁₁ hydrocarbons from methanol. In the process, the educts carbon monoxide and hydrogen are introduced into a reactor 4 via conduit 1 and valve 2 as well as conduit 3.

From the reactor 4, methanol synthesized already together with non-converted educts can be introduced via conduit 5 into a downstream reactor 7, from which the crude methanol obtained together with non-converted educts is recirculated via conduit 6 into the reactor 4, in which it acts as heating medium for the reaction taking place there and is cooled at the same time.

The crude methanol along with remaining, non-converted educts then is withdrawn via conduit 8 and supplied to a high-pressure flash evaporator 11. This high-pressure flash evaporator 11 is operated at 60 to 100 bar. Gaseous components are supplied to the valve 13 via conduit 12. Via conduit 14, parts of the gaseous components are recirculated into the valve 2 by means of a non-illustrated condenser and from there are introduced into the reactor 4 via conduit 3, in order to ensure that still contained carbon monoxide and hydrogen can be converted in the reactor to obtain a valuable product.

Via conduit 15, the remaining rest is supplied to a scrubber 16. This this scrubber 16, washing water is supplied via conduit 17. Via conduit 18 the purified gas is discharged, while the washing water is passed on via conduit 19.

Liquid constituents are discharged from the high-pressure flash evaporator 11 via conduit 20 into a low-pressure flash evaporator 21, which is operated at 1 to 3 bar absolute. From the low-pressure evaporator 21 further gases are supplied to a scrubber 23 via conduit 22. Conduit 19 opens into the scrubber 23, which thus supplies the washing water to the scrubber 23. Via conduit 24, the purified gases are withdrawn. It is, however, also conceivable to supply the two scrubbers 16 and 23 with separate streams of washing water, in order to avoid an entrainment of impurities.

Via conduit 25, the purified liquid fractions are supplied to a distillation column 30. Its bottom product is withdrawn via conduit 31 and substantially consists of methanol. Via conduit 32, the top product is supplied to a heater and heated there. The correspondingly heated product is passed into the condenser 36 via conduit 34. Liquid constituents are recirculated into the distillation column 30 via conduit 37, while via conduit 38 gaseous products are supplied to the second heater 39 from which they are withdrawn via conduit 40.

Via conduit 42, water can be recirculated from the scrubber 23 into the flash evaporator 21.

According to the invention, a feed conduit 41 leads from the scrubber 23 into the condenser 36 and here introduces the liquid water. In the same way, however, the water stream also can be withdrawn already from the scrubber 16, or a separate water stream or a water stream obtained at another point in the process can be fed in. It likewise is conceivable to provide a separate supply for the water at the head of the column or to already charge the water into the heater 33 and thus bring it up to temperature.

By this addition of water, hydrocarbons with a chain length of six to eleven carbon atoms can be separated effectively.

### EXAMPLES

### Example 1

The example shows the comparison between a conventionally operated distillation column and one operated according to the invention. As shown in Table 1, the content of hydrocarbons with a chain length between six and eleven carbon atoms decreases significantly. In the first experiment, a total of 2000 wt-ppm of hydrocarbons (HC) were charged to the feed and in the second experiment a total of 1000 ppm each with 15 vol-% of C₉₊ fractions. In the first experiment, a HC concentration of 240 ppm could be detected in the sump, in the second experiment still 60 ppm. It could be demonstrated that the hydrocarbons in the sump exclusively consist of a C₉₊ fraction. The low-boiling components were separated over head. Thus, the separation efficiency with head injection could be increased to 30% recovery based on the C₉₊ fraction of 80%. In particular, hydrocarbons with 10 C-atoms could be separated significantly better.

TABLE 1
Conditions of the two runs carried out according to Example 1

| | Experiment 1 | Experiment 2 |
|---|---|---|
| Inflow (g/h) | 400 | 400 |
| Water addition at the head (g/h) | — | 12 |
| Water content (% of the inflow) | 5 | 3* |
| Reflux rate | 20 | 20 |
| Head flux (% of the inflow) | 6 | 6 |
| Pressure/bar | 2.3 | 2.3 |
| Hydrocarbons in the feed (ppm (mass)) | 2000 | 1000 |
| C₉₊ fraction of hydrocarbons/% | 15 | 15 |
| Recovery** hydrocarbons/% | 12 | 6 |
| Hydrocarbons fraction in the sump/ppm | 240 | 60 |
| (mass) | | |
| C₉₊ fraction/ppm (mass) | 100 | 100 |
| Recovery** C₉₊/% | 80 | 30 |

*with water added over head also 5% of the inflow.
**Recovery = mass of the component in the sump/mass of the component in the feed

### Example 2

Crude methanol from a plant with pure quality, i.e. high hydrocarbon concentrations, was charged to a distillation column at a height of four meters, with an inner diameter of 50 mm and 20 separation stages. The methanol was introduced continuously at the separation tray 15. The reflux at the head of the column was adjusted such that it was 2% of the methanol inflow.

At first, no water was added (Experiments 1 and 2). By addition 10 vol-% of water to the inflow, the quality of the separated methanol could be increased already (Experiments 3 and 4). By adding such an amount of water at the head of the column that it was 0.56% of the methanol inflow, the specifications of the turbidity test according to Spec O-M-232L could be met (Experiments 5 to 8).

TABLE 2
Experimental conditions with continuous forerun distillation

| | Experiment No. | | | | | | | |
|---|---|---|---|---|---|---|---|---|
| | 1 | 2 | 3 | 4 | 5 | 6 | 7 | 8 |
| Addition of water to crude methanol | — | — | 10 | 10 | — | — | — | — |
| (vol-%) | | | | | | | | |
| Addition of water at the head (g/h) | — | — | — | — | 10 | 10 | 10 | 10 |
| Feed quantity of crude methanol (1/h) | 3.0 | 2.0 | 2.7 | 2.7 | 2.7 | 2.7 | 2.7 | 2.7 |
| Feed quantity of water (1/h) | — | — | 0.3 | 0.3 | 0.3 | 0.3 | 0.3 | 0.3 |
| Top product of the total feed (%) | 2 | 5 | 2 | 2 | 2 | 1 | 1 | 0.5 |
| Top product of the crude methanol (%) | 2 | 5 | 2.2 | 2.2 | 2.2 | 1.1 | 1.1 | 0.56 |
| Boiling rate (W/h) | 100 | 100 | 100 | 133 | 133 | 133 | 133 | 133 |
| Dissipated condensation heat (kcal/h) | 77 | 77 | 77 | 77 | 77 | 77 | 77 | 77 |

For determining the purity, the turbidity of the methanol was measured as significant quantity on a scale from 0 (complete impermeability to light) to 100 (completely clear). The prepurified methanol then was distilled again in a further column for water separation, wherein the above-mentioned volume fraction each was withdrawn over head as pure methanol.

Without addition of water (Experiment 1) hydrocarbons still are found in all fractions, independent of how clearly the separation is made in this downstream column, as here turbidities occur. By addition of water in Experiments 2 and 3 into the inflow, the turbidities could be reduced distinctly. When water is added over head (Experiments 5 to 8), the hydrocarbons obviously are separated even better and a reduction of the top product withdrawn is achieved, as is shown by the reduced turbidity.

TABLE 2
Turbidity values of the fractions from the discontinuous laboratory
distillation; there was each used stabilized methanol from a continuous
distillation

| | Fraction | | | | | |
|---|---|---|---|---|---|---|
| | | | | | 70- | Total |
| | 0-10% | 10-20% | 20-30% | 40-50% | 80% | distillate* |
| Experiment 1 | <50 | 72 | 83 | 94 | — | — |
| Experiment 3 | 76 | 92 | 97 | 98 | 96 | 94 |
| Experiment 4 | 80 | 95 | 98 | 99 | 99 | 96 |
| Experiment 5 | 98 | 100 | 100 | 98 | 98 | 98 |
| Experiment 6 | 99 | 100 | 96 | 99 | 100 | 99 |
| Experiment 8 | 99 | 100 | 100 | 100 | — | 100 |

*Distillation here was carried out until the head temperature had increased by 0.2-0.3° C. above the value constant during the preceding time. This practically corresponds to a complete distillation of the sump.

TABLE 3
Pure-methanol analyses from Experiments 9 and 10 as compared
to a reference.

| | Experiment 9 | | | |
|---|---|---|---|---|
| | (from | Experiment 10 | | |
| | Experiment | (from | | Crude |
| | 7) | Experiment 8) | Reference | MeOH |
| Density | 0.792 | 0.792 | 0.792 | |
| Water (ppm) | 220 | 250 | 200-500 | |
| Boiling interval | 0.45 | 0.2 | 0.35 | |
| (° C.) | | | | |
| Acid (ppm) | 15 | 15 | 15 | |
| Permanganate | 75 | 70 | 35-45 | |
| index (min) | | | | |
| Gas | | | | |
| chromatography | | | | |
| Acetone | 19 | 11 | 13-22 | 51 |
| Methyl formate | 150 | 135 | 120 | 606 |
| Ethanol | 380 | 290 | n.a. | 698 |
| Unknown** | 980 | 1000 | 1040 | 885 |

*A still unknown, presumably higher-boiling substance coincides with methyl formate.
**These are 5 components, two of which constitute the main fraction. Experiment 9 relates to Distillation Experiment No. 7 and Experiment 10 relates to No. 8

### REFERENCE NUMERALS

1 conduit
2 valve
3 conduit
4 reactor
5, 6 conduit
7 reactor
8 conduit
11 flash evaporator
12 conduit
13 valve
14, 15 conduit
16 scrubber
17-20 conduit
21 flash evaporator
22 conduit
23 scrubber
24, 25 conduit
30 distillation column
30a sump (of the distillation column)
30b head (of the distillation column)
31, 32 conduit
33 heater
34 conduit
36 condenser
37, 38 conduit
39 heater
40 conduit
41 feed conduit
42 conduit

1-15. (canceled)

16. A plant for removing C₆-C₁₁ hydrocarbons from methanol, comprising:
at least one reactor for the conversion of carbon monoxide and hydrogen to a crude methanol containing hydrocarbons,
a distillation column with a head and a sump configured to purify the methanol, and
at least one conduit configured to guide the crude methanol from the at least one reactor into the distillation column,
wherein the head of the distillation column includes a feed conduit configured to feed in water to the head of the distillation column.

17. The apparatus according to claim 16, wherein in the at least one conduit for the supply of the crude methanol from the reactor into the distillation column at least one flash evaporator is provided for separating gases from the crude methanol.

18. The apparatus according to claim 17, wherein downstream of the at least one flash evaporator at least one scrubber is provided such that in the at least one scrubber the separated gases are purified by means of a water wash.

19. The apparatus according to claim 18, wherein the feed conduit is designed such that it guides the washing water from the at least one scrubber into the head of the distillation column.

20. The apparatus according to claim 16, wherein the distillation column is designed such that the purified methanol is withdrawn via the sump.

21. The apparatus according to claim 16, wherein in its head the distillation column includes a reflux device with a heater and a condenser and the feed conduit opens into the reflux device.

22. The apparatus according to claim 16, wherein the distillation column is a packed column or contains sieve trays or bubble trays.

23. A process for removing C₆-C₁₁ hydrocarbons from methanol, the process comprising the steps of:
converting carbon monoxide and hydrogen to a crude methanol containing hydrocarbon; and
purifying the crude methanol in a distillation column with a head and a sump, wherein water is added to the head of the column during purification of the crude methanol.

24. The process according to claim 23, wherein 1 to 15% of water based on the total volume flow of inflow and water fed in is added.

25. The process according to claim 23, wherein the contained hydrocarbons contain six to eleven carbon atoms in an amount of at least 50%, preferably 80%, based on the total volume flow of all contained carbon atoms.

26. The process according to claim 23, wherein the crude methanol contains between 90 and 97 vol-% of methanol, between 3 and 10 vol-% of water, up to 0.5 wt-% of hydrocarbons with an amount of C₉₊ fraction of up to 15%, and 0.3 vol-% of further impurities.

27. The process according to claim 23, wherein the distillation column includes a reflux.

28. The process according to claim 27, wherein the reflux ratio lies between 1 and 5.

29. The process according to claim 23, wherein the distillation column is operated at a temperature between 75 and 100° C. and/or at a pressure of 1 to 3 bar.

30. The process according to claim 23, wherein a flux withdrawn over head corresponds to 0.3 to 0.7 vol-% of the inflow into the distillation column.
